(19) United States
(12) Patent Application Publication
(10) Pub. No.: US 2026/0061101 A1
(43) Pub. Date: Mar. 5, 2026

(54) FABRICATED ENGINEERED SKIN DERMIS COMPOSITIONS AND METHODS THEREOF

(71) Applicant: THE TEXAS A&M UNIVERSITY SYSTEM, College Station, TX (US)

(72) Inventors: Feng ZHAO, College Station, TX (US); Dhavan SHARMA, College Station, TX (US)

(21) Appl. No.: 19/101,088

(22) PCT Filed: Aug. 4, 2023
(86) PCT No.: PCT/US2023/029468

§ 371 Date: Feb. 4, 2025

Related U.S. Application Data
(60) Provisional application No. 63/395,488, filed on Aug. 5, 2022.
(60) Provisional application No. 63/441,261, filed on Jan. 26, 2023.

(51) Int. Cl. A61L 27/60; A61F 2/10; A61L 27/38; A61L 27/48

(52) U.S. Cl. CPC A61L 27/60; A61F 2/105; A61L 27/3808; A61L 27/3834; A61L 27/48

(57) ABSTRACT

The present disclosure provides an engineered dermis comprising one or more layers of human dermal fibroblast compositions as well as scaffolds and methods of the same. The disclosure also provides compositions comprising one or more interwoven extracellular matrices, wherein the interwoven extracellular matrices have been decellularized as well as scaffolds and methods of the same.

### CROSS-REFERENCE TO RELATED APPLICATIONS

This application claims the benefit under 35 USC § 119 (e) of U.S. Provisional Application Ser. No. 63/395,488, filed on Aug. 5, 2022, and of U.S. Provisional Application Ser. No. 63/441,261, filed on Jan. 26, 2023. The entire disclosures of both provisional applications to which this application claims benefit are incorporated herein by reference.

### GOVERNMENT RIGHTS

This invention was made with government support under NIH R01HL146652 awarded by the National Institute of Health and under NSF 2106048 awarded by the National Science Foundation. The government has certain rights in the invention.

### BACKGROUND AND SUMMARY OF THE INVENTION

Skin grafts are an essential component for the treatment of several different disease states. For instance, treatment strategies for many kinds of wounds and burns rely on the use of skin grafts to be used on patients.

Diabetic foot ulcers (DFU) and other chronic wounds affect 6.5 million patients per year at a cost burden of $25 billion (USD) on the medical system. A lifetime risk of DFU development among the 30 million diabetic Americans is nearly 25-30% with a recurrence rate ranging from 12%-100% within 12-40 months, respectively. The immediate immune response in non-chronic wounds incorporates production of proinflammatory cytokines, antimicrobial peptides, and recruitment of phagocytic cells, which clear up microbial contaminations. This primary response is followed by changes in the inflammatory status of the immune system, when macrophages switch from the pro-inflammatory (M1) phenotype to the regenerative (M2) phenotype, which advances the wound healing trajectory in the proliferative and finally the remodeling stage.

The macrophage phenotypic switch (M1 to M2) may be a key prerequisite for wound closure. In chronic wounds, healing is arrested in the late inflammatory stage, instead of progressing toward a successful formation of granulation tissue that eventually results in a non-healing ulcer. Factors contributing to pathogenesis of diabetic chronic wounds include (1) inadequate clearance of contamination, (2) unsuccessful granulation tissue formation and keratinocyte proliferation due to inadequate growth factor secretion, (3) a prolonged inflammation secreting proteases that continually breakdown growth factors and extracellular matrix (ECM) fibers.

Moreover, the human dermis contains highly organized and complex ECM architecture that ensures the mechanical stability and elasticity of skin. Although transplanting acellular dermal matrices (ADM) derived from various tissues from xenogeneic or human donor sources have been attempted, natural ADM are problematic due to autologous tissue and organ scarcity, the risk of possible rejection from host immune responses, and pathogen transfer upon the use of allogenic and xenogeneic tissues and organs.

Full thickness skin grafts can provide better texture and softness compared to split-thickness skin grafts, which contains the epidermis and a superficial portion of the dermis. Thus, the flexibility of the skin is believed to be provided by the reticular dermis that includes collagen fibers (type I and type III), elastic fibers and other ground substances. Further, interwoven fibrous structures can increase wound closure within 14 days compared with scaffolds with aligned or random fibers architecture. Unfortunately, none of the current commercial products in the art possess such a structure. Although the ADM DermaACELL® may have an interwoven structure, the gap between ECM fibril is too large and exposes a large area of the wound (few mm² to cm²) and has a risk of undesirable scar formations.

In summary, despite the high demand for skin grafts, there is an urgent need for new compositions that can achieve clinical success and meet the needs of patients. Further complications include biocompatibility challenges associated with donor-based and non-human grafts as well as their inadequate mechanical stability and elasticity. Therefore, there exists a need to develop new engineered skin dermis compositions and methods to address the challenges posed by current practices.

Accordingly, the present disclosure provides an engineered dermis as well as associated methods and compositions thereof. Furthermore, the present disclosure provides compositions comprising one or more interwoven extracellular matrices that can be completely biological and can closely mimic the interwoven ECM architecture and components of native dermis.

As detailed in the present disclosure, several benefits can be realized using the described engineered dermis and associated methods. First, the engineered dermis is obtained from human non-donors and thus are completely biological (e.g., from a biological source, with no syntheitc or chemical components). Second, the engineered dermis is not sourced from a non-human animal such as a bovine. Third, the engineered dermis can be produced in multiple layers to provide desired thickness characteristics. Fourth, the engineered dermis can be utilzied as a scaffold to deliver therapeutics or cells to a desired location in a patient. Fifth, the engineered dermis can fully integrate with the native tissue and undergo remodeling as native tissues. Sixth, the engineered dermis does not contain living cells, which does not require stringent schedule plans or conditions for manufacturing, storage, and transportation. Seventh, the engineered dermis mimic can be configured with an architecture that is substantially similar to native human dermis, for instance by including a native collagen orientation and/or a native elastin orientation, the micro-structure of the vessel walls to provide optimal mechanical properties and orientation cues for endothelialization and remodeling.

Furthermore, several benefits can be realized using the described compositions comprising one or more interwoven extracellular matrices. These compositions can be completely biological and can also closely mimic the interwoven ECM architecture and components of native dermis. As described herein, a micro-pillared silicon wafer using soft-lithography approach than can guide human dermal fibroblast (hDF) growth in order to develop the interwoven hDF sheets was developed. Decellularization resulted in a dermal-specific interwoven ECM (iECM) that is mechanically robust and has a Young's modulus similar to the human skin. Importantly, in vitro co-culturing of macrophages on iECM demonstrated that the iECM is not immunogenic and can stimulate macrophage polarization towards pro-inflammatory phenotype. Therefore, the described compositions comprising one or more interwoven extracellular matrices can be utilized for wound healing applications. Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 shows a histogram illustrating the bimodal distribution of collagen fibers in the superficial (red) and deep reticular layers (blue) using collagen fiber orientation angles in a representative cumulative curve with Gaussian fitting.

FIG. 2A shows a representative high magnified image of the micro-pillar mask design.

FIG. 2B shows a representative magnified image of the micro-pillar mask design.

FIG. 2C shows a representative image of the micro-pillar mask design for the entire 6 cm×6 cm silicon wafer.

FIG. 3A shows a representative images of a micro-pillared PDMS mold casted from silicon wafer and observed via light microscopy (top) and field-emission scanning electron microscopy (FESEM) (bottom).

FIG. 3B shows a representative light microscopy image of hDF cultured on micro-pillared PDMS forming interwoven hDF sheets.

FIG. 3C shows a representative light microscopy image of an interwoven ECM (IECM) developed following decellularization of the interwoven hDF-sheets.

FIG. 4A shows a representative image of Mega hDF-sheet (square, 5 cm×5 cm, are 25 cm²) before decellularization with interwoven architecture.

FIG. 4B shows a representative image of Mega iECM (square, 5 cm×5 cm, are 25 cm²) developed following decellularization of the interwoven hDF-sheets.

FIG. 5A shows a representative image of Mini hDF-sheet (circular, area ˜2 cm²) before decellularization with interwoven architecture.

FIG. 5B shows a representative image of Mini iECM (circular, area ˜2 cm²) developed following decellularization of the interwoven hDF-sheets.

FIG. 6A shows a representative image of IECM easily peeled-off from the base PDMS, without any residual ECM adhered between the micro-pillars.

FIG. 6B shows a representative image of iECM self-sustaining interwoven architecture with 90 degree angles between ECM fiber bundles after its detachment from the base PDMS and folded in half (Scale bar 1 cm).

FIG. 6C shows a representative image of multi-folded (4-folded) iECM self-sustaining interwoven architecture with 90 degree angles between ECM fiber bundles with resulting thickness ˜200 μm (Scale bar 1 cm).

FIG. 7A shows a representative FE-SEM image of hDF-secreted ECM bundles observed surrounding the pillars and organized in an interwoven pattern. Stretch and relaxed ECM fibers observed via red and green arrows, respectively.

FIG. 7B shows a representative FE-SEM magnified image hDF-secreted ECM bundles observed surrounding the pillars (green and red arrows) and organized in an interwoven pattern. Stretch and relaxed ECM fibers observed via red and green arrows, respectively.

FIG. 7C shows a representative FE-SEM higher magnified image of iECM reveal a nano-scale fibrous architecture of ECM bundles.

FIG. 8A shows a stress strain curve obtained from stretching of iECM and rECM sheets.

FIG. 8B shows a representative image of a setup for stretching iECM/rECM sheets using a tensile testing device.

FIG. 8C shows a representative image showing gross morphology of iECM scaffold that was stretched for tensile testing.

FIG. 8D shows a representative image showing gross morphology of rECM scaffold that was stretched for tensile testing.

FIG. 9A shows a bar graph illustrating the mechanical properties comparing iECM and rECM regarding the measurement of modulus (*p<0.05, **p<0.01, ***p<0.001, ****p<0.0001).

FIG. 9B shows a bar graph illustrating the mechanical properties comparing iECM and rECM regarding the measurement of toughness (*p<0.05, **p<0.01, ***p<0.001, ****p<0.0001).

FIG. 9C shows a bar graph illustrating the mechanical properties comparing iECM and rECM regarding the measurement of maximum stress (*p<0.05, **p<0.01, ***p <0.001, ****p<0.0001).

FIG. 9D shows a bar graph illustrating the mechanical properties comparing iECM and rECM regarding the measurement of strain at maximum stress (*p<0.05, **p<0.01, ***p<0.001, ****p<0.0001).

FIG. 10A shows representative confocal images of interwoven hDF-sheet illustrating the interwoven organization of major ECM proteins collagen-I, fibronectin, and laminin (green) surrounding the micro-pillars using immunofluorescence staining. F-actin (red) and DAPI (blue).

FIG. 10B shows representative confocal images of hDF-ECM (iECM) illustrating the interwoven organization of major ECM proteins collagen-I, fibronectin, and laminin (green) surrounding the micro-pillars using immunofluorescence staining. hDF-ECM did not show positive signal for F-actin (red) and DAPI (blue).

FIG. 11A shows representative confocal images with z-stacking illustrating the thickness of interwoven hDF-sheet and hDF-ECM (IECM). Specific structural proteins including collagen-I (green), F-actin (red) and DAPI (blue) were stained.

FIG. 11B shows representative confocal images with z-stacking illustrating the thickness of interwoven hDF-sheet and hDF-ECM (iECM). Specific structural proteins including fibronectin (green), F-actin (red), and DAPI (blue) were stained.

FIG. 11C shows representative confocal images with z-stacking illustrating the thickness of interwoven hDF-sheet and hDF-ECM (iECM). Specific structural proteins including laminin (green), F-actin (red), and DAPI (blue) were stained.

FIG. 12A shows a representative bar graph comparing construct thickness between interwoven hDF-sheet and iECM (after decellularization).

FIG. 12B shows a representative bar graph comparing Pico-green based quantification of DNA concentration in interwoven hDF-sheet (before decellularization) and iECM (after decellularization) (****p<0.0001).

FIG. 13A shows a representative bar graph comparing total collagen from interwoven hDF-sheet (before decellularization) and iECM (after decellularization) (*p<0.05, **p<0.01, ***p<0.001, ****p<0.0001).

FIG. 13B shows a representative bar graph comparing elastin from interwoven hDF-sheet (before decellularization) and iECM (after decellularization) (*p<0.05, **p<0.01, ***p<0.001, ****p<0.0001).

FIG. 13C shows a representative bar graph comparing fibronectin from interwoven hDF-sheet (before decellularization) and iECM (after decellularization) (*p<0.05. **p<0.01, ***p<0.001, ****p<0.0001).

FIG. 13D shows a representative bar graph comparing sGAG from interwoven hDF-sheet (before decellularization) and iECM (after decellularization) (*p<0.05, **p<0.01, ***p<0.001, ****p<0.0001).

FIG. 14A shows a representative pie graph illustrating the most abundant 15 proteins in iECM and rECM based on the intensities of each protein to indicate their abundance within scaffold. Combined intensities of rest of the proteins identified in each ECM were mentioned as sum of the rest of the proteins (indicated via grey color).

FIG. 14B shows a representative pie graph illustrating the most abundant 15 proteins identified in iECM in comparison to their levels with rECM.

FIG. 15A shows representative confocal images of macrophage cultured on iECM and rECM stained with macrophage specific markers (CD68 (green), CD163 (red)), F-actin (magenta) and DAPI (blue) at days 3 and 7 post macrophage seeding (scale bar 100 μm).

FIG. 15B shows a representative bar graph comparing ratio CD163 and CD68 signals obtained from macrophage seeded onto the iECM and rECM at days 3 and 7.

FIG. 16A shows a representative image of a western blot assay illustrating expression of macrophage associated pro-inflammatory and anti-inflammatory markers in macrophage cultured on iECM and rECM. Proteins isolated from M0, M1 and M2 considered as controls.

FIG. 16B shows a representative bar graph illustrating the percentage normalized intensities measured for pro-inflammatory marker CD11b, as determined by a western blot assay.

FIG. 16C shows a representative bar graph illustrating the percentage normalized intensities measured for pro-inflammatory marker GLUT1 as determined by a western blot assay.

FIG. 16D shows a representative bar graph illustrating the percentage normalized intensities measured for pro-inflammatory marker HK2 as determined by a western blot assay.

FIG. 16E shows a representative bar graph illustrating the percentage normalized intensities measured for anti-inflammatory marker CD163 as determined by a western blot assay.

FIG. 16F shows a representative bar graph illustrating the percentage normalized intensities measured for anti-inflammatory marker ARG1 as determined by a western blot assay.

FIG. 16G shows a representative bar graph illustrating the percentage normalized intensities measured for anti-inflammatory marker SIRT1 as determined by a western blot assay.

FIG. 17A shows representative images illustrating the cytokines detected from macrophage cultured on iECM and rECM for 3 and 7 days.

FIG. 17B shows a representative bar graph illustrating the quantification of percentage pixel densities of the detected cytokines. From the array of 36 cytokines, only 6 cytokines were detected from the media and cell lysate collected from macrophage cultured on iECM and rECM.

FIG. 17C shows a representative heat map diagram of the detected cytokines. From the array of 36 cytokines, only 6 cytokines were detected from the media and cell lysate collected from macrophage cultured on iECM and rECM.

FIG. 18 shows representative images illustrating the histological analysis of tissue constructs in the full-thickness wound bed of diabetic Lewis rats. H&E staining evaluates the morphology of iECM and rECM tissue constructs in full-thickness diabetic wound beds (blue arrow). The morphology of iECM and rECM tissue constructs improved over time, with an overall reduction in the wound bed and the presence of neogenetic hair follicles (yellow arrow) observed by day 28. The blue arrow with black dotted demarcated boundaries marked this progress. There was a complete epithelial layer formation mimicking the native skin architecture. The sham group had unhealthy granulation tissue (green arrow), characterized by the presence of dark red cells in the wound bed. No neogenetic hair follicles were observed in any of the dermal layers of the groups. E, D, and H represent Epidermis, Dermis, and Hypodermis, respectively. Scale bar 1 mm.

FIGS. 19A-19C show various iECM design with increased surface area to cover large burn wounds. FIG. 19A shows a “zigzag” pattern. FIG. 19B shows an exemplary micropillar structure that increases the surface area of the entire patch upon its detachment from base PDMS. FIG. 19C shows the exemplary micropillar structure following bi-directional stretching. These exemplary versions of pillar design can provide a patch that can be stretched at least 1.5 times of its original area to satisfy the needs to covering large surface areas in the case of severe burn wounds.

### DETAILED DESCRIPTION

Various embodiments of the invention are described herein as follows. In an illustrative aspect, an engineered dermis is provided. The engineered dermis comprises one or more layers of human dermal fibroblast compositions.

In an embodiment, the engineered dermis comprises an architecture that is substantially similar to native human dermis. In an embodiment, the native human dermis is native reticular dermis. In an embodiment, the architecture comprises a native collagen orientation. In an embodiment, the native collagen orientation comprises an angle of collagen fibers of about 90%. In an embodiment, the architecture comprises a native elastin orientation. In an embodiment, the native elastin orientation comprises an angle of elastin fibers of about 90%.

In an embodiment, the engineered dermis is substantially free of cells. As used herein, the term “substantially free” can refer to a low number or a low concentration, such as less than 5%, less than 4%, less than 3%, less than 2%, less than 1%, less than 0.1%, and the like. In an embodiment, the engineered dermis is substantially free of living cells. In an embodiment, the engineered dermis is acellular.

In an embodiment, the engineered dermis is obtained from human dermis. In an embodiment, the engineered dermis comprises human dermis. In an embodiment, the engineered dermis consists essentially of human dermis. In an embodiment, the engineered dermis consists of human dermis. In an embodiment, the engineered dermis is completely biological. As used herein, biological refers to origin from a biological source for instance with no syntheitc or chemical components. In an embodiment, the engineered dermis does not comprise components from human donors.

In an embodiment, the engineered dermis does not comprise components from a non-human animal. In an embodiment, the non-human animal is a bovine.

In an embodiment, the engineered dermis comprises collagen, elastin, fibronectin, laminin, proteoglycans, or any combination thereof. In an embodiment, the engineered dermis comprises collagen. In an embodiment, the engineered dermis comprises elastin. In an embodiment, the engineered dermis comprises fibronectin. In an embodiment, the engineered dermis comprises laminin. In an embodiment, the engineered dermis comprises proteoglycans. In an embodiment, the engineered dermis is immunocompatible. As used herein, immunocompatible refers to compatibility with the immune system of an animal, for instance a human.

In an embodiment, the layers of human dermal fibroblast compositions are configured in a stacked formation. In an embodiment, the layers of human dermal fibroblast compositions are configured in an interwoven direction. As used herein, interwoven refers to an architecture that is similar to mimics the ECM architecture of human reticular dermis and/or an the interwoven organization of collagen fibrils in skin. In an embodiment, the layers of human dermal fibroblast compositions are decellularized. As used herein, decellularized refers to removal of cells and can be measured, for instance, by the presence of DNA. For example, decellularization can refer to a DNA content less than 10 μg, less than 9 μg, less than 8 μg, less than 7 μg, less than 6 μg, less than 5 μg, less than 4 μg, less than 3 μg, less than 2 μg, or less than 1 μg. In an embodiment, one or more of the decellularized layers of human dermal fibroblast compositions has a total collagen concentration similar to the layers of human dermal fibroblast compositions prior to decellularizaiton. In an embodiment, one or more of the decellularized layers of human dermal fibroblast compositions has an elastin concentration similar to the layers of human dermal fibroblast compositions prior to decellularizaiton.

In an illustrative aspect, a scaffold composition is provided. The scaffold composition comprises the engineered dermis of any of the embodiments of the present disclosure.

In an embodiment, the scaffold composition comprises an additional component for delivery.

In an embodiment, the additional component comprises cells. In an embodiment, the additional component comprises human mesenchymal stem cells (hMSCs). In an embodiment, the additional component comprises endothelial cells (ECs).

In an embodiment, the additional component comprises a cell-derived factor. In an embodiment, the cell-derived factor is selected from the group consisting of exosomes, angiogenic factors, anti-inflammatory factors, micro RNA (miRNA), small interfering RNA (siRNA), or any combination thereof.

In an embodiment, the additional component comprises a chemical entity. In an embodiment, the additional component comprises a drug.

In an illustrative aspect, a method of treating a disorder of a patient is provided. The method comprises the step of administering the engineered dermis of any of the embodiments of the present disclosure to the patient.

In an embodiment, the disorder is a wound. In an embodiment, the wound is an acute wound. In an embodiment, the wound is a chronic wound. In an embodiment, the wound is on a foot of the patient. In an embodiment, the wound is a diabetic foot ulcer. In an embodiment, the disorder is a burn. In an embodiment, the disorder is associated with diabetes. In an embodiment, the disorder is associated with neuropathy. In an embodiment, the disorder is associated with chronic inflammation. In an embodiment, the disorder is associated with reactive oxygen species (ROS). In an embodiment, the disorder is associated with hypoxia. In an embodiment, the disorder is associated with impaired angiogenesis.

In an embodiment, the patient is an immunocompromised patient. In an embodiment, the patient is an elderly patient.

In an illustrative aspect, a method of treating a disorder of a patient is provided. The method comprises the step of administering the scaffold composition of any of the embodiments of the present disclosure to the patient.

In an embodiment, the disorder is a wound. In an embodiment, the wound is an acute wound. In an embodiment, the wound is a chronic wound. In an embodiment, the wound is on a foot of the patient. In an embodiment, the wound is a diabetic foot ulcer.

In an embodiment, the disorder is a burn. In an embodiment, the disorder is associated with diabetes. In an embodiment, the disorder is associated with neuropathy. In an embodiment, the disorder is associated with chronic inflammation. In an embodiment, the disorder is associated with reactive oxygen species (ROS). In an embodiment, the disorder is associated with hypoxia. In an embodiment, the disorder is associated with impaired angiogenesis.

In an embodiment, the patient is an immunocompromised patient. In an embodiment, the patient is an elderly patient.

In an illustrative aspect, a process of fabricating an engineered dermis comprising multiple layers of human dermal fibroblast compositions is provided. The process comprises the step of stacking the layers of human dermal fibroblast compositions on a substrate to form the engineered dermis.

In an embodiment, the substrate is a silicon wafer. In an embodiment, the silicon wafer comprises polydimethylsiloxane. In an embodiment, the silicon wafer consists essentially of polydimethylsiloxane. In an embodiment, the silicon wafer consists of polydimethylsiloxane.

In an embodiment, the substrate comprises a plurality of pillars. In an embodiment, the pillars are micron-scale.

In an embodiment, the layers are interwoven. In an embodiment, the method comprises decellularizing the human dermal fibroblast compositions.

In an illustrative aspect, an engineered dermis comprising multiple layers of human dermal fibroblast compositions formed according to any of the processes described herein is provided.

In an illustrative aspect, a composition comprising one or more interwoven extracellular matrices is provided, wherein the interwoven extracellular matrices have been decellularized.

In an embodiment, the composition is dermal-specific. In an embodiment, the composition comprises a Young's modulus. In an embodiment, the Young's modulus is between 2 Mpa and 10 Mpa. In an embodiment, the Young's modulus is between 2 Mpa and 5 Mpa. In an embodiment, the Young's modulus is between 2 Mpa and 4 Mpa. In an embodiment, the Young's modulus is 2 Mpa. In an embodiment, the Young's modulus is 2.5 Mpa. In an embodiment, the Young's modulus is 3 Mpa. In an embodiment, the Young's modulus is 3.5 Mpa. In an embodiment, the Young's modulus is 4 Mpa. In an embodiment, the Young's modulus is 4.5 Mpa. In an embodiment, the Young's modulus is 5 Mpa. In an embodiment, the Young's modulus is 5.5 Mpa. In an embodiment, the Young's modulus is 6 Mpa. In an embodiment, the Young's modulus is 6.5 Mpa. In an embodiment, the Young's modulus is 7 Mpa. In an embodiment, the Young's modulus is 7.5 Mpa. In an embodiment, the Young's modulus is 8 Mpa. In an embodiment, the Young's modulus is 8.5 Mpa. In an embodiment, the Young's modulus is 9 Mpa. In an embodiment, the Young's modulus is 9.5 Mpa. In an embodiment, the Young's modulus is 10 Mpa. In an embodiment, the composition comprises a Young's modulus similar to human skin.

In an embodiment, the composition comprises a toughness between 100 kJ/m³ and 500 kJ/m³. In an embodiment, the toughness is between 100 kJ/m³ and 200 kJ/m³. In an embodiment, the toughness is between 100 kJ/m³ and 300 kJ/m³. In an embodiment, the toughness is between 100 kJ/m³ and 400 kJ/m³.

In an embodiment, the composition comprises a maximum stress between 300 kPa and 1500 kPa. In an embodiment, the maximum stress is between 300 kPa and 1500 kPa. In an embodiment, the maximum stress is between 500 kPa and 1000 kPa. In an embodiment, the maximum stress is between 700 kPa and 900 kPa. In an embodiment, the maximum stress is between 800 kPa and 900 kPa

In an embodiment, the composition comprises an architecture that is substantially similar to native human dermis. In an embodiment, the composition is substantially free of cells. In an embodiment, the composition is substantially free of living cells. In an embodiment, the composition is acellular.

In an embodiment, the composition is obtained from human dermis. In an embodiment, the composition comprises human dermis. In an embodiment, the composition consists essentially of human dermis. In an embodiment, the composition consists of human dermis. In an embodiment, the composition is completely biological. In an embodiment, the composition does not comprise components from human donors.

In an embodiment, the composition does not comprise components from a non-human animal. In an embodiment, the non-human animal is a bovine.

In an embodiment, the composition comprises collagen, elastin, fibronectin, laminin, proteoglycans, or any combination thereof. In an embodiment, the composition comprises collagen. In an embodiment, the composition comprises elastin. In an embodiment, the composition comprises fibronectin. In an embodiment, the composition comprises laminin. In an embodiment, the composition comprises proteoglycans.

In an embodiment, the composition is immunocompatible. In an embodiment, the composition stimulates macrophage polarization towards pro-inflammatory phenotype.

In an embodiment, the composition comprises one or more tensile properties similar to native dermis. In an embodiment, the one or more tensile properties is mechanical robustness.

In an embodiment, the interwoven extracellular matrices have a total collagen concentration similar to extracellular matrices prior to decellularizaiton. In an embodiment, the interwoven extracellular matrices have an elastin concentration similar to extracellular matrices prior to decellularizaiton.

In an illustrative aspect, a scaffold composition is provided. The scaffold composition comprises the composition of any of the embodiments of the present disclosure.

In an embodiment, the scaffold comprises an additional component for delivery.

In an embodiment, the additional component comprises cells. In an embodiment, the additional component comprises human mesenchymal stem cells (hMSCs). In an embodiment, the additional component comprises endothelial cells (ECs).

In an embodiment, the additional component comprises a cell-derived factor. In an embodiment, the cell-derived factor is selected from the group consisting of exosomes, angiogenic factors, anti-inflammatory factors, micro RNA (miRNA), small interfering RNA (siRNA), or any combination thereof.

In an embodiment, the additional component comprises a chemical entity. In an embodiment, the additional component comprises a drug.

In an illustrative aspect, a method of treating a disorder of a patient is provided. The method comprises the step of administering the composition of any of the embodiments of the present disclosure to the patient.

In an embodiment, the disorder is a wound. In an embodiment, the wound is an acute wound. In an embodiment, the wound is a chronic wound. In an embodiment, the wound is on a foot of the patient. In an embodiment, the wound is a diabetic foot ulcer.

In an embodiment, the disorder is a burn. In an embodiment, the disorder is associated with diabetes. In an embodiment, the disorder is associated with neuropathy. In an embodiment, the disorder is associated with chronic inflammation. In an embodiment, the disorder is associated with reactive oxygen species (ROS). In an embodiment, the disorder is associated with hypoxia. In an embodiment, the disorder is associated with impaired angiogenesis.

In an embodiment, the patient is an immunocompromised patient. In an embodiment, the patient is an elderly patient.

In an illustrative aspect, a method of treating a disorder of a patient is provided.

The method comprises the step of administering the scaffold composition of any of the embodiments of the present disclosure to the patient.

In an embodiment, the disorder is a wound. In an embodiment, the wound is an acute wound. In an embodiment, the wound is a chronic wound. In an embodiment, the wound is on a foot of the patient. In an embodiment, the wound is a diabetic foot ulcer.

In an embodiment, the disorder is a burn. In an embodiment, the disorder is associated with diabetes. In an embodiment, the disorder is associated with neuropathy. In an embodiment, the disorder is associated with chronic inflammation. In an embodiment, the disorder is associated with reactive oxygen species (ROS). In an embodiment, the disorder is associated with hypoxia. In an embodiment, the disorder is associated with impaired angiogenesis.

In an embodiment, the patient is an immunocompromised patient. In an embodiment, the patient is an elderly patient.

In an illustrative aspect, a process of fabricating a composition comprising one or more interwoven extracellular matrices is provided. The process comprises the step of contacting extracellular matrices on a substrate to form the composition.

In an embodiment, the substrate is a silicon wafer. In an embodiment, the silicon wafer comprises polydimethylsiloxane. In an embodiment, the silicon wafer consists essentially of polydimethylsiloxane. In an embodiment, the silicon wafer consists of polydimethylsiloxane.

In an embodiment, the substrate comprises a plurality of pillars. In an embodiment, the pillars are micron-scale.

In an embodiment, the layers are interwoven. In an embodiment, the method comprises decellularizing the interwoven extracellular matrices.

In an illustrative aspect, a composition comprising one or more interwoven extracellular matrices formed according to the process of the present disclosure is provided.

The following numbered embodiments are contemplated and are non-limiting:
1. An engineered dermis comprising one or more layers of human dermal fibroblast compositions.
2. The engineered dermis of clause 1, any other suitable clause, or any combination of suitable clauses, wherein the engineered dermis comprises an architecture that is substantially similar to native human dermis.
3. The engineered dermis of clause 2, any other suitable clause, or any combination of suitable clauses, wherein the native human dermis is native reticular dermis.
4. The engineered dermis of clause 2, any other suitable clause, or any combination of suitable clauses, wherein the architecture comprises a native collagen orientation.
5. The engineered dermis of clause 4, any other suitable clause, or any combination of suitable clauses, wherein the native collagen orientation comprises an angle of collagen fibers of about 90%.
6. The engineered dermis of clause 2, any other suitable clause, or any combination of suitable clauses, wherein the architecture comprises a native elastin orientation.
7. The engineered dermis of clause 6, any other suitable clause, or any combination of suitable clauses, wherein the native elastin orientation comprises an angle of elastin fibers of about 90%.
8. The engineered dermis of clause 1, any other suitable clause, or any combination of suitable clauses, wherein the engineered dermis is substantially free of cells.
9. The engineered dermis of clause 1, any other suitable clause, or any combination of suitable clauses, wherein the engineered dermis is substantially free of living cells.
10. The engineered dermis of clause 1, any other suitable clause, or any combination of suitable clauses, wherein the engineered dermis is acellular.
11. The engineered dermis of clause 1, any other suitable clause, or any combination of suitable clauses, wherein the engineered dermis is obtained from human dermis.
12. The engineered dermis of clause 1, any other suitable clause, or any combination of suitable clauses, wherein the engineered dermis comprises human dermis.
13. The engineered dermis of clause 1, any other suitable clause, or any combination of suitable clauses, wherein the engineered dermis consists essentially of human dermis.
14. The engineered dermis of clause 1, any other suitable clause, or any combination of suitable clauses, wherein the engineered dermis consists of human dermis.
15. The engineered dermis of clause 1, any other suitable clause, or any combination of suitable clauses, wherein the engineered dermis is completely biological.
16. The engineered dermis of clause 1, any other suitable clause, or any combination of suitable clauses, wherein the engineered dermis does not comprise components from human donors.
17. The engineered dermis of clause 1, any other suitable clause, or any combination of suitable clauses, wherein the engineered dermis does not comprise components from a non-human animal.
18. The engineered dermis of clause 17, any other suitable clause, or any combination of suitable clauses, wherein the non-human animal is a bovine.
19. The engineered dermis of clause 1, any other suitable clause, or any combination of suitable clauses, wherein the engineered dermis comprises collagen, elastin, fibronectin, laminin, proteoglycans, or any combination thereof.
20. The engineered dermis of clause 1, any other suitable clause, or any combination of suitable clauses, wherein the engineered dermis comprises collagen.
21. The engineered dermis of clause 1, any other suitable clause, or any combination of suitable clauses, wherein the engineered dermis comprises elastin.
22. The engineered dermis of clause 1, any other suitable clause, or any combination of suitable clauses, wherein the engineered dermis comprises fibronectin.
23. The engineered dermis of clause 1, any other suitable clause, or any combination of suitable clauses, wherein the engineered dermis comprises laminin.
24. The engineered dermis of clause 1, any other suitable clause, or any combination of suitable clauses, wherein the engineered dermis comprises proteoglycans.
25. The engineered dermis of clause 1, any other suitable clause, or any combination of suitable clauses, wherein the engineered dermis is immunocompatible.
26. The engineered dermis of clause 1, any other suitable clause, or any combination of suitable clauses, wherein the layers of human dermal fibroblast compositions are configured in a stacked formation.
27. The engineered dermis of clause 1, any other suitable clause, or any combination of suitable clauses, wherein the layers of human dermal fibroblast compositions are configured in an interwoven direction.
28. The engineered dermis of clause 27, any other suitable clause, or any combination of suitable clauses, wherein the layers of human dermal fibroblast compositions are decellularized.
29. The engineered dermis of clause 28, any other suitable clause, or any combination of suitable clauses, wherein one or more of the decellularized layers of human dermal fibroblast compositions has a total collagen concentration similar to the layers of human dermal fibroblast compositions prior to decellularizaiton.
30. The engineered dermis of clause 28, any other suitable clause, or any combination of suitable clauses, wherein one or more of the decellularized layers of human dermal fibroblast compositions has an elastin concentration similar to the layers of human dermal fibroblast compositions prior to decellularizaiton.
31. A scaffold composition comprising the engineered dermis of any of clauses 1 to 30.
32. The scaffold composition of clause 31, any other suitable clause, or any combination of suitable clauses, wherein the scaffold composition comprises an additional component for delivery.
33. The scaffold composition of clause 32, any other suitable clause, or any combination of suitable clauses, wherein the additional component comprises cells.
34. The scaffold composition of clause 32, any other suitable clause, or any combination of suitable clauses, wherein the additional component comprises human mesenchymal stem cells (hMSCs).
35. The scaffold composition of clause 32, any other suitable clause, or any combination of suitable clauses, wherein the additional component comprises endothelial cells (ECs).
36. The scaffold composition of clause 32, any other suitable clause, or any combination of suitable clauses, wherein the additional component comprises a cell-derived factor.
37. The scaffold composition of clause 36, any other suitable clause, or any combination of suitable clauses, wherein the cell-derived factor is selected from the group consisting of exosomes, angiogenic factors, anti-inflammatory factors, micro RNA (miRNA), small interfering RNA (siRNA), or any combination thereof.
38. The scaffold composition of clause 32, any other suitable clause, or any combination of suitable clauses, wherein the additional component comprises a chemical entity.
39. The scaffold composition of clause 32, any other suitable clause, or any combination of suitable clauses, wherein the additional component comprises a drug.
40. A method of treating a disorder of a patient, said method comprising the step of administering the engineered dermis of any of clauses 1 to 30 to the patient.
41. The method of clause 40, any other suitable clause, or any combination of suitable clauses, wherein the disorder is a wound.
42. The method of clause 41, any other suitable clause, or any combination of suitable clauses, wherein the wound is an acute wound.
43. The method of clause 41, any other suitable clause, or any combination of suitable clauses, wherein the wound is a chronic wound.
44. The method of clause 41, any other suitable clause, or any combination of suitable clauses, wherein the wound is on a foot of the patient.
45. The method of clause 41, any other suitable clause, or any combination of suitable clauses, wherein the wound is a diabetic foot ulcer.
46. The method of clause 40, any other suitable clause, or any combination of suitable clauses, wherein the disorder is a burn.
47. The method of clause 40, any other suitable clause, or any combination of suitable clauses, wherein the disorder is associated with diabetes.
48. The method of clause 40, any other suitable clause, or any combination of suitable clauses, wherein the disorder is associated with neuropathy.
49. The method of clause 40, any other suitable clause, or any combination of suitable clauses, wherein the disorder is associated with chronic inflammation.
50. The method of clause 40, any other suitable clause, or any combination of suitable clauses, wherein the disorder is associated with reactive oxygen species (ROS).
51. The method of clause 40, any other suitable clause, or any combination of suitable clauses, wherein the disorder is associated with hypoxia.
52. The method of clause 40, any other suitable clause, or any combination of suitable clauses, wherein the disorder is associated with impaired angiogenesis.
53. The method of clause 40, any other suitable clause, or any combination of suitable clauses, wherein the patient is an immunocompromised patient.
54. The method of clause 40, any other suitable clause, or any combination of suitable clauses, wherein the patient is an elderly patient.
55. A method of treating a disorder of a patient, said method comprising the step of administering the scaffold composition of any of clauses 30 to 39 to the patient.
56. The method of clause 55, any other suitable clause, or any combination of suitable clauses, wherein the disorder is a wound.
57. The method of clause 56, any other suitable clause, or any combination of suitable clauses, wherein the wound is an acute wound.
58. The method of clause 56, any other suitable clause, or any combination of suitable clauses, wherein the wound is a chronic wound.
59. The method of clause 56, any other suitable clause, or any combination of suitable clauses, wherein the wound is on a foot of the patient.
60. The method of clause 56, any other suitable clause, or any combination of suitable clauses, wherein the wound is a diabetic foot ulcer.
61. The method of clause 55, any other suitable clause, or any combination of suitable clauses, wherein the disorder is a burn.
62. The method of clause 55, any other suitable clause, or any combination of suitable clauses, wherein the disorder is associated with diabetes.
63. The method of clause 55, any other suitable clause, or any combination of suitable clauses, wherein the disorder is associated with neuropathy.
64. The method of clause 55, any other suitable clause, or any combination of suitable clauses, wherein the disorder is associated with chronic inflammation.
65. The method of clause 55, any other suitable clause, or any combination of suitable clauses, wherein the disorder is associated with reactive oxygen species (ROS).
66. The method of clause 55, any other suitable clause, or any combination of suitable clauses, wherein the disorder is associated with hypoxia.
67. The method of clause 55, any other suitable clause, or any combination of suitable clauses, wherein the disorder is associated with impaired angiogenesis.
68. The method of clause 55, any other suitable clause, or any combination of suitable clauses, wherein the patient is an immunocompromised patient.
69. The method of clause 55, any other suitable clause, or any combination of suitable clauses, wherein the patient is an elderly patient.
70. A process of fabricating an engineered dermis comprising multiple layers of human dermal fibroblast compositions, said process comprising the step of stacking the layers of human dermal fibroblast compositions on a substrate to form the engineered dermis.
71. The process of clause 70, any other suitable clause, or any combination of suitable clauses, wherein the substrate is a silicon wafer.
72. The process of clause 71, any other suitable clause, or any combination of suitable clauses, wherein the silicon wafer comprises polydimethylsiloxane.
73. The process of clause 71, any other suitable clause, or any combination of suitable clauses, wherein the silicon wafer consists essentially of polydimethylsiloxane.
74. The process of clause 71, any other suitable clause, or any combination of suitable clauses, wherein the silicon wafer consists of polydimethylsiloxane.
75. The process of clause 70, any other suitable clause, or any combination of suitable clauses, wherein the substrate comprises a plurality of pillars.
76. The process of clause 75, any other suitable clause, or any combination of suitable clauses, wherein the pillars are micron-scale.
77. The process of clause 70, any other suitable clause, or any combination of suitable clauses, wherein the layers are interwoven.
78. The process of clause 70, any other suitable clause, or any combination of suitable clauses, wherein the method comprises decellularizing the human dermal fibroblast compositions.
79. An engineered dermis comprising multiple layers of human dermal fibroblast compositions formed according to the process of any one of clauses 70 to 78.
80. A composition comprising one or more interwoven extracellular matrices, wherein the interwoven extracellular matrices have been decellularized.
81. The composition of clause 80, any other suitable clause, or any combination of suitable clauses, wherein the composition is dermal-specific.
82. The composition of clause 80, any other suitable clause, or any combination of suitable clauses, wherein the composition comprises a Young's modulus.
83. The composition of clause 82, any other suitable clause, or any combination of suitable clauses, wherein the Young's modulus is between 2 Mpa and 10 Mpa.
84. The composition of clause 82, any other suitable clause, or any combination of suitable clauses, wherein the Young's modulus is between 2 Mpa and 5 Mpa.
85. The composition of clause 82, any other suitable clause, or any combination of suitable clauses, wherein the Young's modulus is between 2 Mpa and 4 Mpa.
86. The composition of clause 82, any other suitable clause, or any combination of suitable clauses, wherein the Young's modulus is 2 Mpa.
87. The composition of clause 82, any other suitable clause, or any combination of suitable clauses, wherein the Young's modulus is 2.5 Mpa.
88. The composition of clause 82, any other suitable clause, or any combination of suitable clauses, wherein the Young's modulus is 3 Mpa.
89. The composition of clause 82, any other suitable clause, or any combination of suitable clauses, wherein the Young's modulus is 3.5 Mpa.
90. The composition of clause 82, any other suitable clause, or any combination of suitable clauses, wherein the Young's modulus is 4 Mpa.
91. The composition of clause 82, any other suitable clause, or any combination of suitable clauses, wherein the Young's modulus is 4.5 Mpa.
92. The composition of clause 82, any other suitable clause, or any combination of suitable clauses, wherein the Young's modulus is 5 Mpa.
93. The composition of clause 82, any other suitable clause, or any combination of suitable clauses, wherein the Young's modulus is 5.5 Mpa.
94. The composition of clause 82, any other suitable clause, or any combination of suitable clauses, wherein the Young's modulus is 6 Mpa.
95. The composition of clause 82, any other suitable clause, or any combination of suitable clauses, wherein the Young's modulus is 6.5 Mpa.
96. The composition of clause 82, any other suitable clause, or any combination of suitable clauses, wherein the Young's modulus is 7 Mpa.
97. The composition of clause 82, any other suitable clause, or any combination of suitable clauses, wherein the Young's modulus is 7.5 Mpa.
98. The composition of clause 82, any other suitable clause, or any combination of suitable clauses, wherein the Young's modulus is 8 Mpa.
99. The composition of clause 82, any other suitable clause, or any combination of suitable clauses, wherein the Young's modulus is 8.5 Mpa.
100. The composition of clause 82, any other suitable clause, or any combination of suitable clauses, wherein the Young's modulus is 9 Mpa.
101. The composition of clause 82, any other suitable clause, or any combination of suitable clauses, wherein the Young's modulus is 9.5 Mpa.
102. The composition of clause 82, any other suitable clause, or any combination of suitable clauses, wherein the Young's modulus is 10 Mpa.
103. The composition of clause 80, any other suitable clause, or any combination of suitable clauses, wherein the composition comprises a Young's modulus similar to human skin.
104. The composition of clause 80, any other suitable clause, or any combination of suitable clauses, wherein the composition comprises a toughness between 100 kJ/m³ and 500 kJ/m³.
105. The composition of clause 104, any other suitable clause, or any combination of suitable clauses, wherein the toughness is between 100 kJ/m³ and 200 kJ/m³.
106. The composition of clause 104, any other suitable clause, or any combination of suitable clauses, wherein the toughness is between 100 kJ/m³ and 300 kJ/m³.
107. The composition of clause 104, any other suitable clause, or any combination of suitable clauses, wherein the toughness is between 100 kJ/m³ and 400 kJ/m³.
108. The composition of clause 80, any other suitable clause, or any combination of suitable clauses, wherein the composition comprises a maximum stress between 300 kPa and 1500 kPa.
109. The composition of clause 108, any other suitable clause, or any combination of suitable clauses, wherein the maximum stress is between 300 kPa and 1500 kPa.
110. The composition of clause 108, any other suitable clause, or any combination of suitable clauses, wherein the maximum stress is between 500 kPa and 1000 kPa.
111. The composition of clause 108, any other suitable clause, or any combination of suitable clauses, wherein the maximum stress is between 700 kPa and 900 kPa.
112. The composition of clause 108, any other suitable clause, or any combination of suitable clauses, wherein the maximum stress is between 800 kPa and 900 kPa.
113. The composition of clause 80, any other suitable clause, or any combination of suitable clauses, wherein the composition comprises an architecture that is substantially similar to native human dermis.
114. The composition of clause 80, any other suitable clause, or any combination of suitable clauses, wherein the composition is substantially free of cells.
115. The composition of clause 80, any other suitable clause, or any combination of suitable clauses, wherein the composition is substantially free of living cells.
116. The composition of clause 80, any other suitable clause, or any combination of suitable clauses, wherein the composition is acellular.
117. The composition of clause 80, any other suitable clause, or any combination of suitable clauses, wherein the composition is obtained from human dermis.
118. The composition of clause 80, any other suitable clause, or any combination of suitable clauses, wherein the composition comprises human dermis.
119. The composition of clause 80, any other suitable clause, or any combination of suitable clauses, wherein the composition consists essentially of human dermis.
120. The composition of clause 80, any other suitable clause, or any combination of suitable clauses, wherein the composition consists of human dermis.
121. The composition of clause 80, any other suitable clause, or any combination of suitable clauses, wherein the composition is completely biological.
22. The composition of clause 80, any other suitable clause, or any combination of suitable clauses, wherein the composition does not comprise components from human donors.
123. The composition of clause 80, any other suitable clause, or any combination of suitable clauses, wherein the composition does not comprise components from a non-human animal.
124. The composition of clause 123, any other suitable clause, or any combination of suitable clauses, wherein the non-human animal is a bovine.
125. The composition of clause 80, any other suitable clause, or any combination of suitable clauses, wherein the composition comprises collagen, elastin, fibronectin, laminin, proteoglycans, or any combination thereof.
126. The composition of clause 80, any other suitable clause, or any combination of suitable clauses, wherein the composition comprises collagen.
127. The composition of clause 80, any other suitable clause, or any combination of suitable clauses, wherein the composition comprises elastin.
128. The composition of clause 80, any other suitable clause, or any combination of suitable clauses, wherein the composition comprises fibronectin.
129. The composition of clause 80, any other suitable clause, or any combination of suitable clauses, wherein the composition comprises laminin.
130. The composition of clause 80, any other suitable clause, or any combination of suitable clauses, wherein the composition comprises proteoglycans.
131. The composition of clause 80, any other suitable clause, or any combination of suitable clauses, wherein the composition is immunocompatible.
132. The composition of clause 80, any other suitable clause, or any combination of suitable clauses, wherein the composition stimulates macrophage polarization towards pro-inflammatory phenotype.
133. The composition of clause 80, any other suitable clause, or any combination of suitable clauses, wherein the composition comprises one or more tensile properties similar to native dermis.
134. The composition of clause 133, any other suitable clause, or any combination of suitable clauses, wherein the one or more tensile properties is mechanical robustness.
135. The composition of clause 80, any other suitable clause, or any combination of suitable clauses, wherein the interwoven extracellular matrices have a total collagen concentration similar to extracellular matrices prior to decellularizaiton.
136. The composition of clause 80, any other suitable clause, or any combination of suitable clauses, wherein the interwoven extracellular matrices have an elastin concentration similar to extracellular matrices prior to decellularizaiton.
137. A scaffold composition comprising the composition of any of clauses 80 to 136.
138. The scaffold composition of clause 137, any other suitable clause, or any combination of suitable clauses, wherein the scaffold comprises an additional component for delivery.
139. The scaffold composition of clause 138, any other suitable clause, or any combination of suitable clauses, wherein the additional component comprises cells.
140. The scaffold composition of clause 138, any other suitable clause, or any combination of suitable clauses, wherein the additional component comprises human mesenchymal stem cells (hMSCs).
141. The scaffold composition of clause 138, any other suitable clause, or any combination of suitable clauses, wherein the additional component comprises endothelial cells (ECs).
142. The scaffold composition of clause 138, any other suitable clause, or any combination of suitable clauses, wherein the additional component comprises a cell-derived factor.
143. The scaffold composition of clause 142, any other suitable clause, or any combination of suitable clauses, wherein the cell-derived factor is selected from the group consisting of exosomes, angiogenic factors, anti-inflammatory factors, micro RNA (miRNA), small interfering RNA (siRNA), or any combination thereof.
144. The scaffold composition of clause 138, any other suitable clause, or any combination of suitable clauses, wherein the additional component comprises a chemical entity.
145. The scaffold composition of clause 138, any other suitable clause, or any combination of suitable clauses, wherein the additional component comprises a drug.
146. A method of treating a disorder of a patient, said method comprising the step of administering the composition of any of clauses 80 to 136 to the patient.
147. The method of clause 146, any other suitable clause, or any combination of suitable clauses, wherein the disorder is a wound.
148. The method of clause 147, any other suitable clause, or any combination of suitable clauses, wherein the wound is an acute wound.
149. The method of clause 147, any other suitable clause, or any combination of suitable clauses, wherein the wound is a chronic wound.
150. The method of clause 147, any other suitable clause, or any combination of suitable clauses, wherein the wound is on a foot of the patient.
151. The method of clause 147, any other suitable clause, or any combination of suitable clauses, wherein the wound is a diabetic foot ulcer.
152. The method of clause 146, any other suitable clause, or any combination of suitable clauses, wherein the disorder is a burn.
153. The method of clause 146, any other suitable clause, or any combination of suitable clauses, wherein the disorder is associated with diabetes.
54. The method of clause 146, any other suitable clause, or any combination of suitable clauses, wherein the disorder is associated with neuropathy.
155. The method of clause 146, any other suitable clause, or any combination of suitable clauses, wherein the disorder is associated with chronic inflammation.
156. The method of clause 146, any other suitable clause, or any combination of suitable clauses, wherein the disorder is associated with reactive oxygen species (ROS).
157. The method of clause 146, any other suitable clause, or any combination of suitable clauses, wherein the disorder is associated with hypoxia.
158. The method of clause 146, any other suitable clause, or any combination of suitable clauses, wherein the disorder is associated with impaired angiogenesis.
159. The method of clause 146, any other suitable clause, or any combination of suitable clauses, wherein the patient is an immunocompromised patient.
160. The method of clause 146, any other suitable clause, or any combination of suitable clauses, wherein the patient is an elderly patient.
161. A method of treating a disorder of a patient, said method comprising the step of administering the scaffold composition of any of clauses 137 to 145 to the patient.
162. The method of clause 161, any other suitable clause, or any combination of suitable clauses, wherein the disorder is a wound.
163. The method of clause 162, any other suitable clause, or any combination of suitable clauses, wherein the wound is an acute wound.
164 The method of clause 162, any other suitable clause, or any combination of suitable clauses, wherein the wound is a chronic wound.
165. The method of clause 162, any other suitable clause, or any combination of suitable clauses, wherein the wound is on a foot of the patient.
166. The method of clause 162, any other suitable clause, or any combination of suitable clauses, wherein the wound is a diabetic foot ulcer.
167. The method of clause 161, any other suitable clause, or any combination of suitable clauses, wherein the disorder is a burn.
168. The method of clause 161, any other suitable clause, or any combination of suitable clauses, wherein the disorder is associated with diabetes.
169. The method of clause 161, any other suitable clause, or any combination of suitable clauses, wherein the disorder is associated with neuropathy.
170. The method of clause 161, any other suitable clause, or any combination of suitable clauses, wherein the disorder is associated with chronic inflammation.
171. The method of clause 161, any other suitable clause, or any combination of suitable clauses, wherein the disorder is associated with reactive oxygen species (ROS).
172. The method of clause 161, any other suitable clause, or any combination of suitable clauses, wherein the disorder is associated with hypoxia.
173. The method of clause 161, any other suitable clause, or any combination of suitable clauses, wherein the disorder is associated with impaired angiogenesis.
0.74. The method of clause 161, any other suitable clause, or any combination of suitable clauses, wherein the patient is an immunocompromised patient.
175. The method of clause 161, any other suitable clause, or any combination of suitable clauses, wherein the patient is an elderly patient.
176. A process of fabricating a composition comprising one or more interwoven extracellular matrices, said process comprising the step of contacting extracellular matrices on a substrate to form the composition.
177. The process of clause 176, any other suitable clause, or any combination of suitable clauses, wherein the substrate is a silicon wafer.
178. The process of clause 177, any other suitable clause, or any combination of suitable clauses, wherein the silicon wafer comprises polydimethylsiloxane.
179. The process of clause 177, any other suitable clause, or any combination of suitable clauses, wherein the silicon wafer consists essentially of polydimethylsiloxane.
180. The process of clause 177, any other suitable clause, or any combination of suitable clauses, wherein the silicon wafer consists of polydimethylsiloxane.
181. The process of clause 176, any other suitable clause, or any combination of suitable clauses, wherein the substrate comprises a plurality of pillars.
182. The process of clause 181, any other suitable clause, or any combination of suitable clauses, wherein the pillars are micron-scale.
183. The process of clause 176, any other suitable clause, or any combination of suitable clauses, wherein the layers are interwoven.
184. The process of clause 176, any other suitable clause, or any combination of suitable clauses, wherein the method comprises decellularizing the interwoven extracellular matrices.
185. A composition comprising one or more interwoven extracellular matrices formed according to the process of any one of clauses 176 to 184.

The use of the term “or” in the claims and clauses is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

### Example 1

### Exemplary Experimental Procedures

The instant example provides exemplary materials and methods utilized in Examples 2 to 7 as described herein.

Interwoven and Isotropic hDF-ECM Fabrication

A silicon wafer with square micro-pits structure (micro-pit width/length: 500 μm, distance between pits: 200 μm, depth: 170 μm) was prepared with soft lithography technique at the AggieFab nanofabrication facility at Texas A&M University. Photomasks were designed in K-layout software and were custom made and purchased from CAD/Art Services, Inc. for the soft-lithography. Polydimethylsiloxane (PDMS) molds were cased from the silicon wafer using Sylgard 184 Silicone Elastomer Kit (Dow Corning, Midland, MI) by mixing base and cross-linker at 10:1 ratio followed by heating at 65° C. for 4 hours in order to make micro-pillars. Flat PDMS casted from a flat plastic petri dish. Micro-pillared PDMS and flat PDMS were coated with polydopamine and collagen-I prior to cell culture. Briefly, PDMS were immersed in 0.01% W/V 3-hydroxytyramine hydrochloride (Dopamine-HCl) (ACROS Organics, Fisher scientific) for 24 hours followed by ethylene oxide sterilization. Polydopamine coated PDMS were immersed in bovine collagen (20 μg/mL) (Sigma Aldrich, St. Louis, MO) for 2 hours before cell seeding. Neonatal human dermal fibroblasts (ATCC, Manassas, VA) (passage 3-5) were cultured on micropatterned and flat PDMS for 5 weeks to develop anisotropic or isotropic hDF sheets, which were then decellularized to fabricate anisotropic or isotropic hDF-ECM. Briefly, interwoven or isotropic hDF-sheets were decellularized using 1M sodium chloride (NaCl), 0.5% Sodium dodecyl sulfate (SDS), 10 mM Tris and 5 mM ethylenediaminetetraacetate (EDTA) to develop interwoven hDF-ECM (IECM) or randomly organized hDF-ECM (rECM) sheets.

### Evaluation of Mechanical Properties Via Tensile Testing

Elongation tensile testing: Tensile Young's modulus of the hydrogels was determined by making rectangular iECM/rECM sheets with dimensions 25 mm×6.25 mm×200 μm (length×width×thickness) and performing tensile testing using Instron 5944 Universal Testing System (Instron Test Systems, Norwood, MA) equipped with tensile clamps. Specimens were strained at a rate of 1 mm/min. From the resulting stress-strain curves, Young's modulus (E) was calculated from the following equation:

$$E = σ / ε$$

where, E is the young's modulus; σ is the uniaxial force per unit area and ε is strain or the proportional deformation.

### Field Emission Scanning Electron Microscopy

Micro-pillared PDMS and iECM samples were prepared for field emission scanning electron microscopy (FE-SEM). Briefly, iECM sheets were thoroughly rinsed with PBS and were fixed with 10% formalin solution for 24 hours at 40° C. Crosslinked samples were thoroughly washed with PBS and immersed under series of ethanol solutions with 10%, 30%, 50%, 70%, 90% and 100% concentrations. Following dehydration, samples were desiccated overnight, coated with platinum-palladium alloy (Pt—Pd) using a Cressington 208 HR sputter coater (Ted Pella Inc., Redding, CA) and observed under FEI QUANTA 600 FE-SEM (Hillsboro, OR).

### Immunofluorescence Staining

Major ECM protein components of interwoven hDF-sheet and iECM were observed via Immunofluorescence staining (IF). Briefly, samples were fixed in 4% formaldehyde solution and blocked with 1% bovine serum albumin (BSA). Samples were incubated overnight at 4° C. with primary antibody solutions with 1:200 dilution prepared in 1% BSA. Primary antibodies targeting major ECM components (collagen-I, fibronectin, and elastin) or macrophage-specific markers (CD68 and CD163) were used. Samples were stained with secondary antibodies goat-anti mouse Alexa Fluor™ 488 conjugate, goat-anti rabbit Alexa Fluor™ 555 conjugate, and Alexa Fluor™ 647 Phalloidin at 1:200 dilution prepared in 1% BSA. Cell nuclei were stained with 4′,6-diamidino-2-phenylindole, dihydrochloride (DAPI). Color thresholding function in ImageJ software was used to quantify CD68 and CD163 signals from macrophage cultured on iECM and rECM.

Characterization of iECM Construct

Quantification of DNA content in iECM post decellularization was measured using Quant-iT™ PicoGreen™ dsDNA assay kit (Thermo Fisher) and compared with interwoven hDF-sheets. Major ECM components of iECM including total collagen, fibronectin, elastin, and sulfated glucosamine glycans (sGAG) were quantified and compared with interwoven hDF-sheets using commercially available kits and following manufacturer's instructions. Collagen was quantified using Total Collagen Assay Kit (Novus Biologicals). Elastin was quantified using Fastin™ Elastin Assay (Biocolor, Carrickfergus, United Kingdom). Fibronectin was quantified using Human Fibronectin DuoSet ELISA (R&D systems). Sulfated glycosaminoglycan (sGAG) was measured via Blyscan Glycosaminoglycan Assay (Biocolor, Carrickfergus, United Kingdom).

### LC-MS Based Compositional Evaluation of ECM Sheets

LC-MS based compositional analysis of ECM sheets was performed by Creative Proteomics (Shirley, NY). Three ECM (n=3) sheets from each group (iECM and rECM) were digested by trypsin, identified and quantified by applying nano LC-MS/MS platform comprising Ultimate 3000 nano UHPLC system coupled with a Q Exactive HF mass spectrometer (Thermo Fisher Scientific, USA) with an ESI nanospray source. Detailed methods with sample preparation, nano LC-MS/MS Analysis and data analysis are provided in the supplementary file.

### Macrophage Co-Culture on Tissue Constructs

THP-1 monocytes (ATCC) were cultured with RPMI-1640 media (ATCC) supplemented with 10% fetal bovine serum (FBS) (R&D systems) and 1% penicillin/streptomycin (Thermo Fisher). THP-1 monocytes were treated with freshly prepared Phorbol 12-myristate 13-acetate (PMA, Sigma Aldrich) (50 ng/mL) supplemented RPMI-1640 media for 24 hours to promote their differentiation into macrophage. Upon 24 hours of PMA stimulation, almost all THP-1 cells become adherent. To study macrophage response in vitro, THP-1 derived macrophages were trypsinized and seeded onto the iECM or rECM constructs with seeding density of 10⁵ cells/cm² in the incubator with 5% CO₂. PMA-1 induced THP-1 cells were treated with 100 ng/mL of lipopolysaccharide (LPS) and 15 ng/ml of interferon-gamma (IFN-γ) for 24 hours to promote their differentiation into proinflammatory (M1-like) phenotype. PMA-1 induced THP-1 cells were treated with 20 ng/ml of IL-4 for 24 hours to promote their differentiation into anti-inflammatory (M2-like) phenotype.

### Western Blotting

The tissue lysate from macrophage cultured on iECM and rECM for 3 and 7 days were prepared in NP-40 lysis buffer (Thermo Fisher) supplemented with EDTA-free Protease Inhibitor (Sigma Aldrich) and Halt™ Phosphatase Inhibitor Cocktail (Thermo Fisher Scientific) according to the manufacturer's instructions. Denatured cell lysates were separated by sodium dodecyl sulfate-polyacrylamide gel electrophoresis (SDS-PAGE) and transferred onto a polyvinylidene fluoride (PVDF) membrane. PVDF membranes were blocked using Intercept® (PBS) Blocking Buffer (LI-COR) followed by overnight incubation in primary antibody with 1:1000 dilution at 4° C. The membranes were stained using IRDye® 800CW goat anti-rabbit and IRDye® 680LT goat anti-mouse secondary antibodies (LI-COR) with 1:5000 dilution for 1 hour at room temperature (RT) and imaged with Odyssey® clx imaging system (LI-COR).

### Cytokine Array Analysis

The tissue lysate from macrophage cultured on iECM and rECM for 3 and 7 days were prepared in lysis buffer 17 (R&D systems) supplemented with 10 μg/mL Aprotinin (Tocris Bioscience, Ellisville, MO), 10 μg/mL Leupeptin (Tocris Bioscience) and 10 μg/mL Pepstatin (Tocris Bioscience). 100 μg of tissue lysates as well as 250 μl of conditioned media from macrophage cultured on iECM and rECM post 3 and 7 days of macrophage seeding were analyzed using Proteome Profiler Human Cytokine Array Kit (ARY005B, R&D systems) following manufacturer's instructions. The membranes were stained using IRDye-800 CW streptavidin antibody (LI-COR) and imaged with Odyssey® clx imaging system (LI-COR).

### Histological Analysis of Tissue Constructs

Full-thickness wounds were created in diabetic Lewis rats and skin tissue were collected on days 3, 7, 14, and 28, respectively. Each rat was surgically implanted with two engineered tissue constructs, namely interwoven dermal matrix (iECM) and random dermal matrix (rECM). Five rats were used for each group. A portion of the skin from another random area from the back was also harvested and designed as a sham/control. Hematoxylin and eosin (H&E) staining was performed to evaluate the comprehensive depiction of the microanatomy of the tissues from day 3 to day 28.

### Statistical Analysis

Statistical comparisons between experimental groups (sample size n=4) were performed by unpaired t-test using GraphPad Prism software. For imaged based analysis of macrophage polarization, signal intensities from six non-overlapped field were measured and statistical analysis was performed via one-way ANOVA and Tukey's post hoc test using GraphPad Prism software. Results were considered statistically significant for *p<0.05, **p<0.01, ***p<0.001, ****p<0.0001.

### Example 2

### Design and Fabrication of iECM

In the instant example, a completely biological, acellular, and mechanically strong off-the-shelf wound patch (iECM) was developed. iECM doesn't rely upon autologous, allogenic, or xenogeneic sources and can be easily screened for pathogens to prevent undesirable inflammatory and immunological reactions.

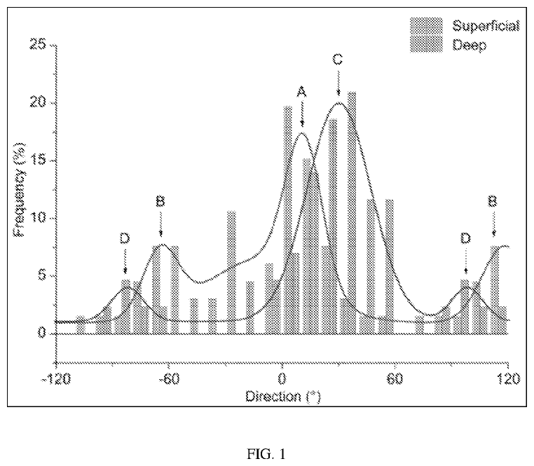

Without being bound by any theory, full thickness skin grafts may provide better texture and softness compared to split-thickness skin grafts and are preferentially used in the reconstruction surgery involving high functional body parts such as joints, face, and neck. This may indicate that the flexibility of the skin may be provided by the reticular dermis that majorly consist of collagen fibers (type I and type III), elastic fibers and other ground substances. Histological studies have indicated that the reticular collagen has a preferred orientation, however, in contrast, SEM imaging indicated collagen architecture has random organization. Therefore, geometric organization of ECM fibers in skin dermis is a controversial subject due to the lack of standardized methods for quantification. To resolve this, previous studies utilized a combination of multiphoton imaging (MPM), tissue clearing method, and biaxial tissue extension to quantitatively analyze the geometry of collagen and elastin fibers in the full thickness skin samples derived from lateral upper thigh of healthy adults (65.5±12.7 years) with no skin diseases or trauma. In MPM technique, second harmonic generation (SHG) mode and two-photon excited autofluorescence (TPAF) were used for label-free visualization of collagen and elastin bundles, respectively. Two-three modes of distributions were found on the collagen fiber architecture in the superficial and deep reticular dermis, which were observed in the previous studies as peaks in the Gaussian fitting (FIG. 1). The angle difference between the peaks was nearly 90 degrees in reticular dermis.

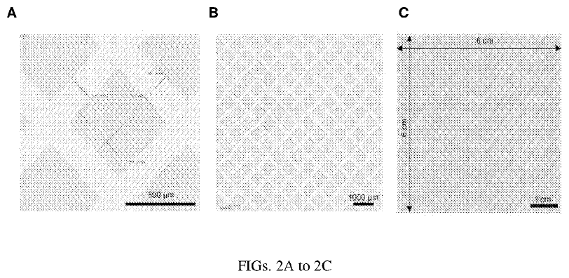

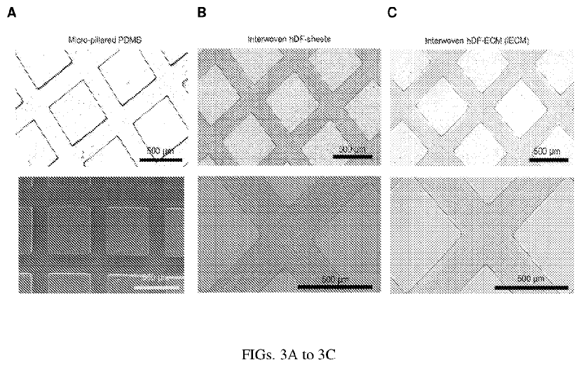

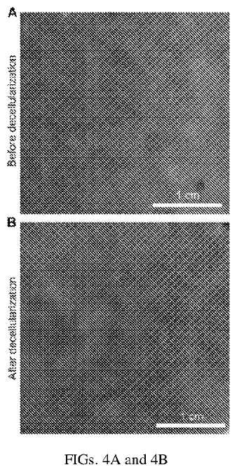

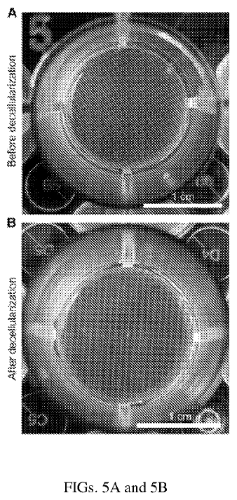

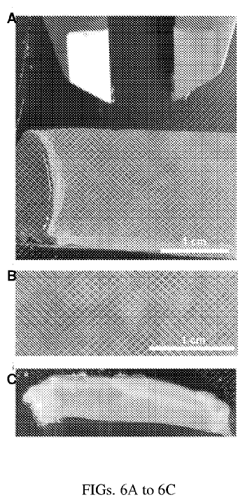

In the instant example, the micro-pillar pattern containing square pillar structure (width and length: 500 μm, distance between the pillars 200 μm) was designed with K-layout software in order to make photomasks (FIGS. 2A, 2B, 2C). The silicon wafer containing micro-pit structures was prepared using soft-lithography to mimic this native collagen orientation (˜90 degree angle) in the engineered acellular dermal matrices (ADM). It was then used as a cast to create PDMS molds with 170 μm-high micro-pillars, as observed via light microscopy and FESEM (FIG. 3A). Upon 5 weeks of culture, hDFs on the micro-pillared PDMS grew into an interwoven hDF-sheet. The low and high magnification light microscopy revealed that hDFs mainly grew in between the pillars and very few cells on the top of the pillars (FIG. 3B). Following decellularization, a dermal-specific interwoven ECM (IECM) was developed, which demonstrated a similar architecture. The iECM nanofibers were observed mostly in between the pillars as observed via light microscopy (FIG. 3C). Without being bound by any theory, iECM in varied sizes and shapes may be prepared by controlling the base PDMS size. As a proof of concept, Mega iECM (square, 5 cm×5 cm, area 25 cm²) (FIG. 4B) and Mini iECM (circular, area ˜2 cm²) (FIG. 5B) were developed, which may be used according to the wound size. Importantly, the iECM has a robust self-sustaining nanofibrous architecture. The iECM maintains its interwoven architecture (with 90° angle) after being peeled off from the base PDMS substrates and folded into multiple layers (FIGS. 6A, 6B, 6C), without retaining any ECM residues adhered to the base PDMS substrate (FIG. 6A). Accordingly and without being bound by any theory, the interwoven design may provide iECM a self-sustainable structure that maintains its ECM-fiber architecture even when folded in multiple layers.

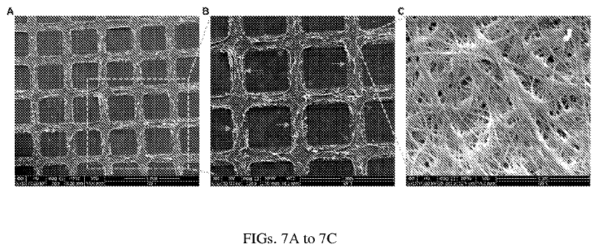

Decellularized iECM were detached from the base micro-pillared PDMS substrates and were imaged with FE-SEM. The FE-SEM images revealed that the iECM formed large ECM bundles in the areas surrounding the pillars (FIG. 7A). High-magnification FE-SEM images revealed the iECM's architecture at sub-micro scale, where tightly packed hDF-ECM fibers organized in an interwoven fashion were observed with diameter in the range of few hundred nanometers. ECM bundles showed alignment in-between the pillars and were observed as in stretched condition at the pillar corners (red arrows, FIG. 7B) but in relaxed condition in between the pillars (green arrows, FIG. 7B). Higher-resolution images revealed that the iECM comprises nano-fibrous bundles (FIG. 7C).

### Example 3

### Characterization of Mechanical Properties

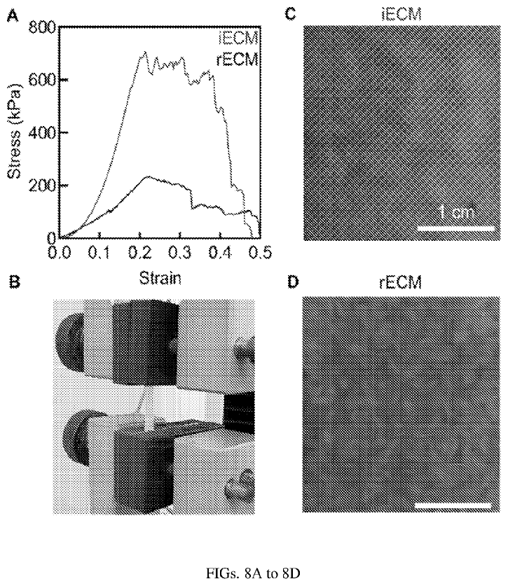

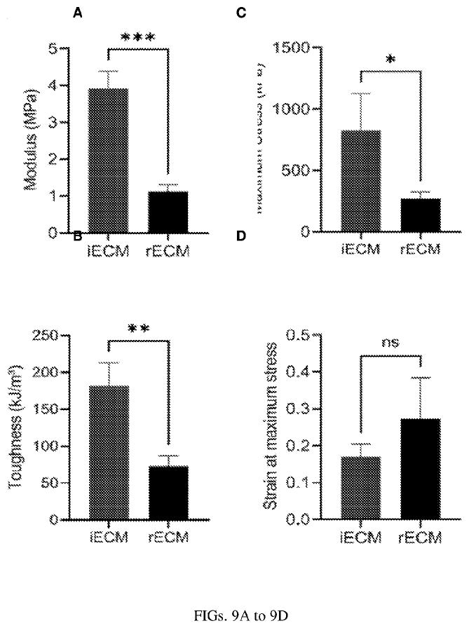

In the instant example, the tensile strength of iECM were analyzed using a tensile stretching device (FIG. 8B) and were compared with rECM. Besides providing self-sustaining architecture, interwoven fiber bundles of iECM also contributed to the tensile strength of the structure. Thick ECM bundles provided iECM with significantly increased modulus and toughness compared to rECM. Stress-strain curve (FIG. 8A) obtained after stretching of iECM (FIG. 8C) and rECM (FIG. 8D) revealed that both ECM scaffolds have amorphous property with plastic nature. Multiple peaks observed during tensile stretching of iECM and rECM sheets indicated a possible re-arrangement of ECM fibers upon stretching, resulting in a cycle of stress build-up and relaxation upon fiber-rearrangement. iECM displayed (3.91+0.47 MPa) significantly higher young's modulus compared with rECM (1.12+0.18 MPa) (p<0.001) (FIG. 9A). Similarly, iECM (181.87+31.21 kJ/m³) had significantly increased toughness compared to rECM (73.05+13.84 kJ/m³), as measured via calculating the area underneath the stress-strain curve (FIG. 9B) (p<0.01). Detected maximum stress was significantly higher in iECM (826.05+300.37 kPa) compared with rECM (273.32+51.17 kPa) (FIG. 9C) (p<0.05). The detected strain at maximum stress was higher in rECM (0.27+0.11) compared to iECM (0.17+0.03) without significant difference (FIG. 9D). Without being bound by any theory, the tensile properties of iECM may indicate its mechanical robustness and modulus in the range of native dermis.

### Example 4

### Evaluation of iECM Structural Components

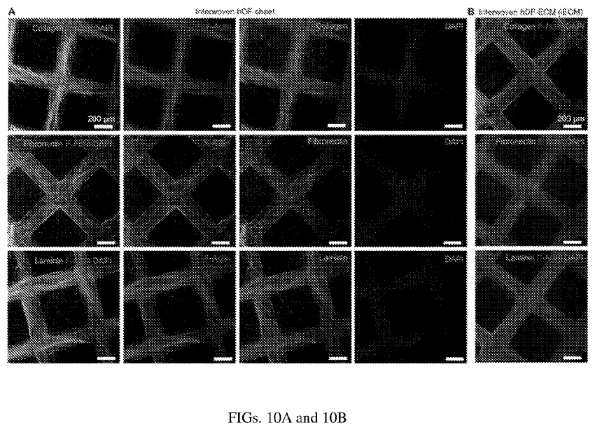

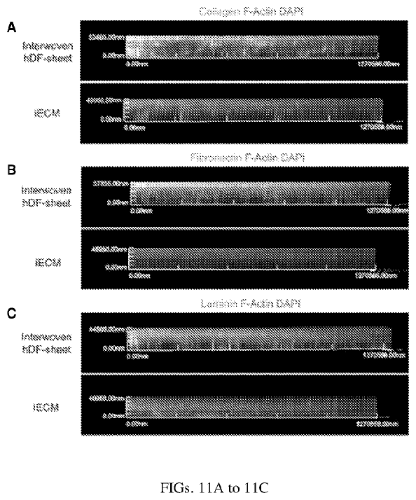

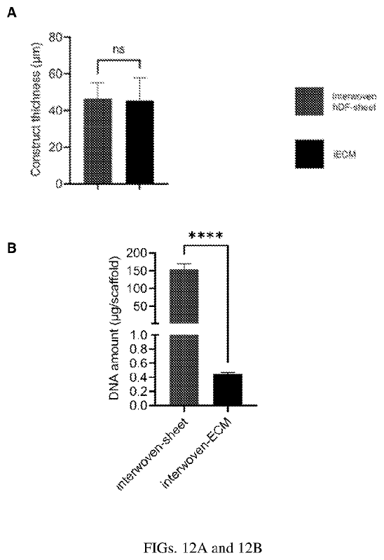

In the instant example, the morphology of key ECM structural components (collagen-I, fibronectin, laminin) were evaluated and compared between the interwoven hDF-sheet and iECM using immunofluorescence staining and confocal imaging. Immunofluorescence (IF) staining revealed an interwoven orientation of collagen I, elastin, fibronectin in hDF-sheet and iECM. (FIGS. 10A, 10B). F-actin and DAPI signals were only observed in interwoven hDF-sheet (FIG. 10A), and not in iECM (FIG. 10B), indicating a successful decellularization and removal of cellular contents. Consistent with the light microscopy (FIGS. 3B, 3C) and FE-SEM (FIGS. 7A, 7B, 7C) imaging results, the interwoven hDF-sheet and iECM were organized into thick bundles mostly between the pillars and not on pillar tops. To determine the thickness of interwoven hDF sheet and iECM, confocal imaging with z-stacking was performed (FIGS. 11A, 11B, 11C). Signals obtained from three major ECM components, collagen-I, fibronectin and laminin, were used to determine the overall thickness of the interwoven hDF-sheet and the iECM in z-stacking. The thickness of iECM was measured as 45.49±12.54 μm (FIG. 12A). No significant difference between the thickness of interwoven hDF-sheet (46.48±8.65 μm) and iECM (45.49±12.54 μm) was observed (FIG. 12A). Pico-green assay-based DNA quantification revealed that iECM had significant reduction in DNA concentration (3.93±0.88 μg/scaffold) compared with interwoven hDF-sheet (15.39±1.63 μg/scaffold), indicating a successful decellularization (FIG. 12B) (p<0.0001). No DNAse or RNAse treatment was provided during the decellularization, however an additional DNAse or RNAse treatment could be included to further reduce the DNA concentration below 3.93±0.88 μg/scaffold.

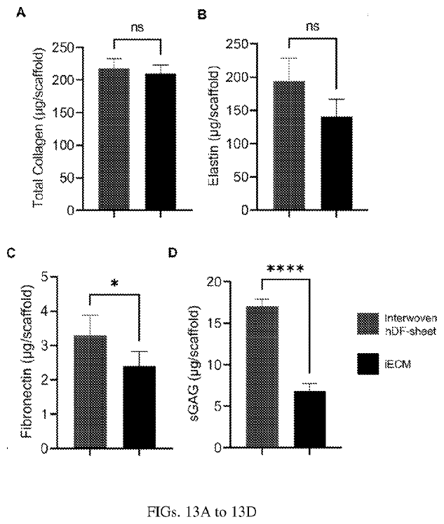

Major ECM-specific macromolecules including total collagen, elastin, fibronectin and sGAG were quantified in iECM and compared with non-decellularized interwoven hDF-sheets. No significant change in total collagen was observed after decellularization in iECM (209.86±13.58 μg/scaffold) compared to interwoven hDF-sheet (217.27±15.58 μg/scaffold) (FIG. 13A). Following decellularization, iECM showed a non-significant reduction in elastin (140.86±26.02 μg/scaffold) compared to interwoven hDF-sheets (193.92±34.76 μg/scaffold) (FIG. 13B). A significant reduction in fibronectin was observed following decellularization in iECM (2.39±0.43 μg/scaffold) compared with interwoven hDF-sheets (3.30±0.58 μg/scaffold) (p<0.05) (FIG. 13C). Similarly, a significant reduction in sGAG was observed following decellularization in iECM (6.86±0.89 μg/scaffold) compared with interwoven hDF-sheets (17.02±0.88 μg/scaffold) (p<0.0001) (FIG. 13D). Without being bound by any theory, it may be possible that comparably thinner fibronectin fibers (˜5-25 nm) could be more susceptible for degradation post-decellularization compared with thicker collagen and elastin fibers. It has been reported that the decellularization solutions containing detergents may result in removal of the GAGs chains from the proteoglycans (PG) core and their possible degradation via β-elimination reaction, resulting in significantly reduced levels of sGAG in iECM.

### Example 5

### LC-MS-Based Compositional Evaluation

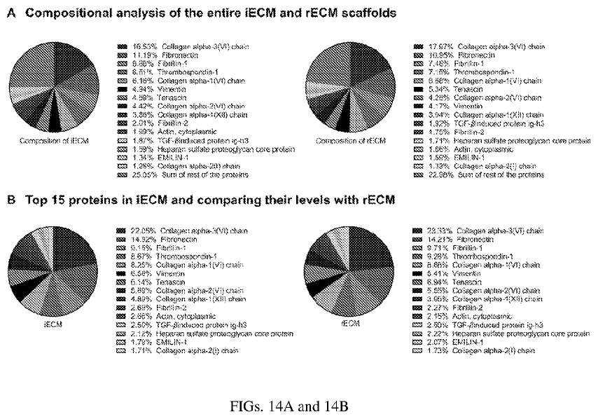

In the instant example, a total of 1486 proteins were identified. Proteins of relative quantitation were divided into two categories. Fold-change (FC)>1.5 was considered up-regulation, while FC<0.67 (½) was considered as down-regulation. Overall, while comparing iECM with rECM, 23 proteins were up regulated (FC>1.5, p<0.05) and 56 proteins were down regulated (FC<1/1.5, p<0.05). Proteins intensities were used as a proxy to determine protein abundance in each ECM type. Without being bound by any theory, LC-MS analysis confirmed that similar protein intensities in both iECM and rECM indicate the micro-pillar structure has no negative effect in altering the expression of ECM proteins. FIG. 14A shows a compositional analysis of iECM and rECM with a list of most abundant 15 proteins in both scaffolds and the combined intensities of the rest of the proteins identified in each ECM, as mentioned as sum of the rest of the proteins. Moreover, the intensities of the most abundant proteins in iECM were compared with rECM, which showed a similar compositional profile of iECM and rECM (FIG. 14B).

### Example 6

### Evaluation of Macrophage Response Toward Interwoven Constructs

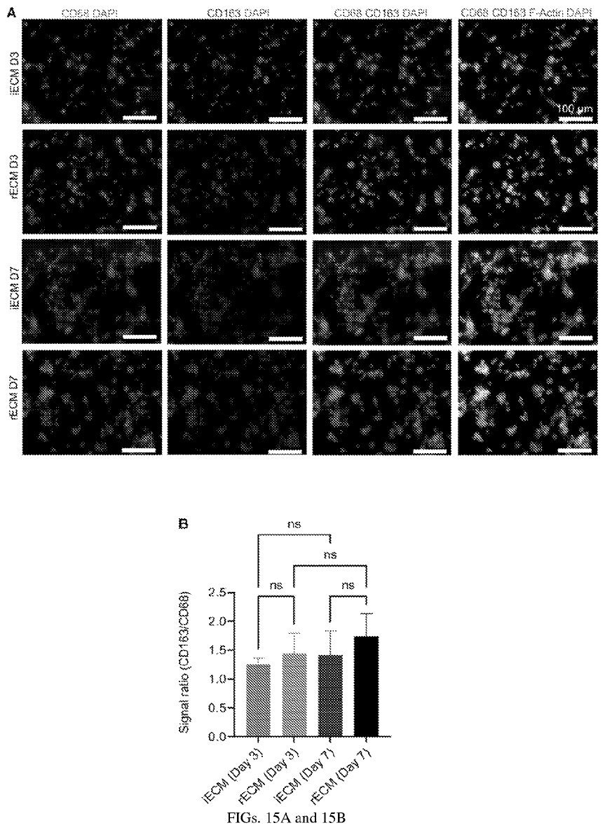

In the instant example, the immunogenicity of iECM constructs were determined via seeding PMA stimulated THP-1 macrophages for 1 week. The macrophage polarization post seeding onto the iECM were evaluated and compared with isotropic/random hDF-ECM sheets (rECM) at days 3 and 7 (FIGS. 15A, 15B). The macrophage seeded iECM and rECM constructs were stained with macrophage specific markers CD68 and CD163, F-actin and DAPI (FIG. 15A). CD163 is a well-established marker for the anti-inflammatory phenotype (M2-like) of macrophage. A ratio of CD163 and CD68 signal was taken to determine the M2-polarization of macrophages on iECM and rECM at days 3 and 7 post macrophage seeding. rECM showed a slightly increased CD163/CD68 ratio compared to iECM at both, day 3 and day 7 (FIG. 15B). However, no significant difference in CD163/CD68 signal ratio was observed between iECM and rECM at days 3 and 7 (FIG. 15B).

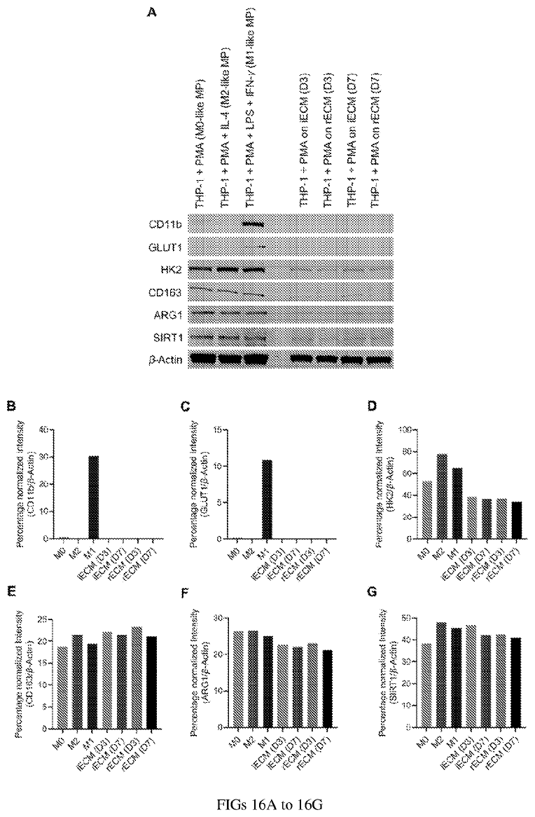

The phenotypic expression of pro-inflammatory and anti-inflammatory phases associated markers of macrophages cultured on iECM and rECM for 3 and 7 days were evaluated via western blotting (FIGS. 16A-16G). Proteins isolated from PMA stimulated macrophage (M0-MP), LPS and IFN-stimulated pro-inflammatory M1-like macrophage and IL-4 stimulated anti-inflammatory M2-like macrophage were also tested as controls, to compare and clearly evaluate metabolic status of macrophage cultured on iECM and rECM (FIG. 16A). The expression of pro-inflammatory markers CD11b and GLUT1 were present in M1-macropahges but were absent in M2 macrophage, as well as in iECM and rECM groups (FIGS. 16B, 16C). Although HK2 is considered as a pro-inflammatory marker, the expression of HK2 was higher in M2 macrophage compared with M1 and M0 macrophage (FIG. 16D). However, both iECM and rECM (days 3 and 7) groups showed reduced expression of HK2 compared with the control M0, M1 and M2 macrophages (FIG. 16D). Unlike M1 markers, macrophages cultured on iECM and rECM showed a clear expression of M2 markers CD163, ARG1 and SIRT1 (FIGS. 16E, 16F, 16G). iECM and rECM groups showed increased expression of anti-inflammatory marker CD163 compared with M2 and M0 macrophages (FIG. 16E). No difference in ARG1 expression was observed between iECM and rECM samples at days 3 and 7 (FIG. 16F). However, the ARG1 expression in iECM and rECM samples were lower compared with M2 macrophages (FIG. 16F). Similar as the M2 macrophages, the day 3 iECM group showed a higher expression of SIRT1 than the day 3 rECM group and the day 7 iECM and rECM groups (FIG. 16G). Increased SIRT1 and ARG1 (involved in l-arginine metabolism) expression has been observed in tumor-associated macrophages that have immunosuppressive and tumorigenic functions.

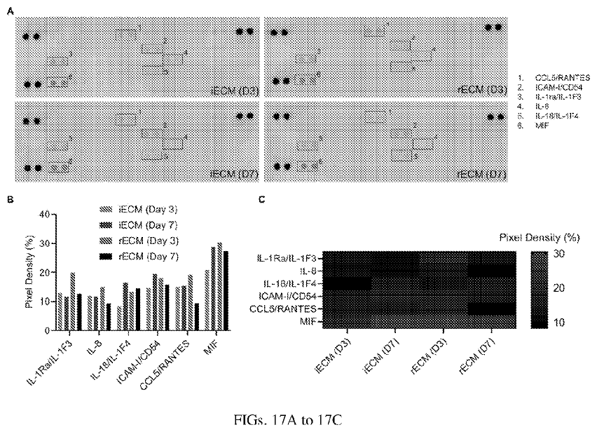

To evaluate the immunogenicity of iECM and rECM in a comprehensive manner, cell lysates and conditioned media isolated from macrophage cultured on iECM and rECM at day 3 and 7 were analyzed using a human cytokine array (FIGS. 17A, 17B, 17C). From the array containing 36 detectable cytokines, pixel densities of only 6 cytokines were detected (FIGS. 17A, 17B). Quantification of percentage pixel density revealed that MIF was highly expressed among the 6 detected cytokines. The day 3 iECM group showed reduced levels of IL-1, IL-8, IL-18, ICAM-1, CCL5/RNATES and MIF than the day 3 rECM (FIGS. 17B, 17C). Levels of IL-1 and IL-8 were reduced form days 3 to day 7 in both, iECM and rECM groups indicating a possible reduction in the pro-inflammatory phenotype of cultured macrophages (FIGS. 17B, 17C).

Among the 6 detected cytokines/surface receptor proteins, CCL-5/RANTES and IL-8 are chemotactic markers secreted by macrophage to recruit leukocytes to the site of inflammation. ICAM-1 is a one of the predominant receptors found on the surface of antigen presenting cells including macrophage that activates MHC class II restricted subtypes of T-cells. Besides these pro-inflammatory cytokines, three anti-inflammatory cytokines IL-1Ra, IL-18 and MIF were detected. IL-1Ra binds with IL-1 receptor and blocks the activity of IL-1A and IL-1B, both prominent pro-inflammatory cytokines involved in myriad of inflammatory mechanisms. Lymphokine MIF functions as a pleiotropic protein, participating in delayed hypersensitivity and affecting key macrophage functions including phagocytosis, spreading, and anti-tumor activities. IL-18 predominantly secreted by macrophage (APCs including macrophage) and is a pleiotropic factor involved in the regulation of both innate and acquired immune responses, playing a key role in autoimmune, inflammatory, and infectious diseases. A recent study revealed that IL-18, in synergy with IL-10 increases osteopontin and thrombin that acts through integrins α4 and α9 to augmenting M2 polarization of macrophage with characteristic CD163 expression. Among all detected cytokines/surface receptors, iECM and ECM showed highest secretion of MIF (FIG. 17B). Overall, without being bound by any theory, cytokine array membranes may display minimal expressions of only 6 cytokines/surface receptor proteins markers, among which no clear trend in the expression of pro- or anti-inflammatory was observed among iECM and rECM groups at day 3 and 7 (FIGS. 17B, 17C). Accordingly and without being bound by any theory, no severe inflammatory response was observed from macrophage cultured on iECM.

### Example 7

### Histological Analysis of Tissue Constructs

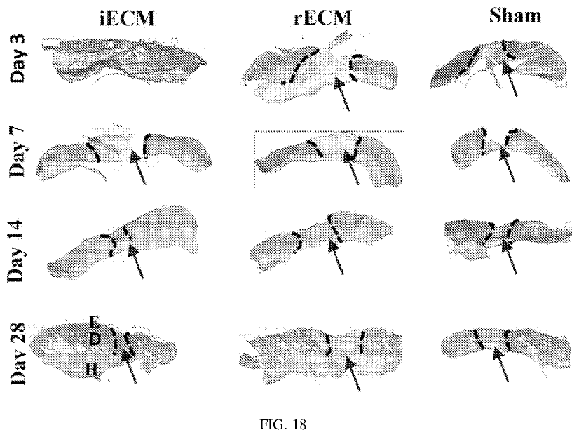

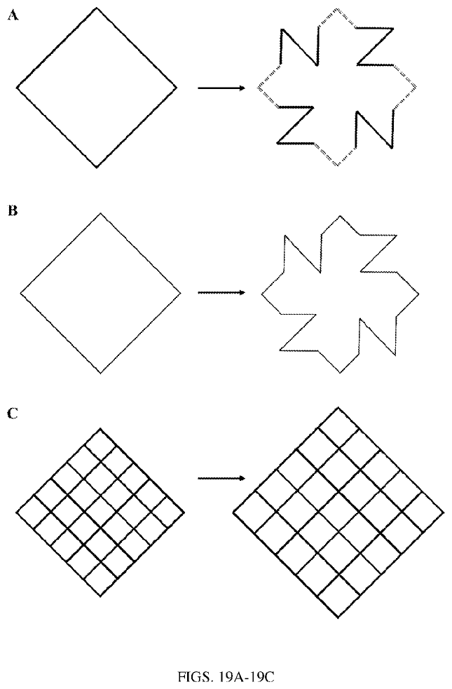

In the instant example, the microanatomy of tissues from full-thickness wounds created in diabetic Lewis rats and skin tissue was evaluated by hematoxylin and eosin (H&E) staining. The normal architecture of rat skin comprises three layers-epidermis (E), dermis (D), and hypodermis (H). The epidermal layer consists of proliferative basal cells that replace dead cells. The dermis layer provides elasticity and contains various microstructures like blood vessels, hair follicles, nerves, and fibers. The hypodermis layer bas collagen and adipose tissue, which is a major source of energy. The regenerated wound bed had an extracellular matrix (ECM) similar to normal skin architecture, and the hypodermis had grown abundantly by day 28. The histological staining showed complete morphological changes in tissue section throughout the study (FIG. 18). On the third day, the wound bed in all three groups was surrounded by cells, wound exudates, and inflammatory mediators. Without being bound by any theory, this differed significantly from the normal skin architecture. The inflammatory cells may be generated by the rat's body to provide antibacterial properties and create an environment conducive to wound repair. Unhealthy granulation tissue (green arrow) was observed in the sham group, characterized by the presence of dark red cells in the wound bed. Additionally, no neogenetic hair follicles in any of the dermal layers of the groups were observed. By the seventh day, tissue growth was evident in all groups, with granulation tissue formation observed in the deeper skin layers of the rat wound bed. A reduction in the wound bed (blue arrow) and fewer inflammatory markers was observed, with the iECM group exhibiting the most significant improvement. By day 14, the epithelial skin layer on the surface of the regenerated wound bed was mostly covered in the iECM group. Less epidermal formation was observed in the rECM and sham groups, and some neogenetic hair follicles were observed in the regenerated wound bed. On the twenty eighth day, complete epidermal layer formation was observed, and the fresh dermis was no longer exposed to air. The regenerated wound bed had an ECM similar to the normal skin architecture, and the hypodermis had grown abundantly. Although there were areas without hair follicles (yellow arrow) due to scar formation, the iECM group had more mature neogenetic hair follicles than the rECM and sham groups.

1. An engineered dermis comprising one or more layers of human dermal fibroblast compositions, wherein the engineered dermis comprises an architecture that is substantially similar to native human dermis.

2. The engineered dermis of claim 1, wherein the native human dermis is native reticular dermis.

3. The engineered dermis of claim 1, wherein the architecture comprises a native collagen orientation.

4. The engineered dermis of claim 3, wherein the native collagen orientation comprises an angle of collagen fibers of about 90°.

5. The engineered dermis of claim 1, wherein the architecture comprises a native elastin orientation.

6. The engineered dermis of claim 5, wherein the native elastin orientation comprises an angle of elastin fibers of about 90°.

7. The engineered dermis of claim 1, wherein the engineered dermis is substantially free of cells.

8. The engineered dermis of claim 1, wherein the engineered dermis is substantially free of living cells.

9. The engineered dermis of claim 1, wherein the engineered dermis is acellular.

10. The engineered dermis of claim 1, wherein the engineered dermis is obtained from human dermis.

11. (canceled)

12. (canceled)

13. (canceled)

14. The engineered dermis of claim 1, wherein the engineered dermis is completely biological.

15. The engineered dermis of claim 1, wherein the engineered dermis does not comprise components from human donors.

16. The engineered dermis of claim 1, wherein the engineered dermis does not comprise components from a non-human animal.

17. (canceled)

18. The engineered dermis of claim 1, wherein the layers of human dermal fibroblast compositions are configured in an interwoven direction.

19. The engineered dermis of claim 18, wherein the layers of human dermal fibroblast compositions are decellularized.

20. A scaffold composition comprising the engineered dermis of claim 1, wherein the scaffold composition comprises an additional component for delivery.

21. (canceled)

22. The scaffold composition of claim 20, wherein the additional component comprises cells.

23. The scaffold composition of claim 20, wherein the additional component comprises human mesenchymal stem cells (hMSCs).

24. The scaffold composition of claim 20, wherein the additional component comprises endothelial cells (ECs).

25. The scaffold composition of claim 20, wherein the additional component comprises a cell-derived factor.

26.-44. (canceled)
